(19) United States
(12) Patent Application Publication
(10) Pub. No.: US 2018/0058987 A1
(43) Pub. Date: Mar. 1, 2018

(54) SYSTEMS AND METHODS FOR SELECTING CELLULAR STRAINS

(71) Applicant: NEW YORK UNIVERSITY, New York, NY (US)

(72) Inventors: Kenneth D. BIRNBAUM, Brooklyn, NY (US); Keith ARORA-WILLIAMS, New York, NY (US)

(21) Appl. No.: 15/560,310

(22) PCT Filed: Mar. 21, 2016
(86) PCT No.: PCT/US16/23449

§ 371 Date: Sep. 21, 2017

Related U.S. Application Data
(60) Provisional application No. 62/137,093, filed on Mar. 23, 2015.

(51) Int. Cl. G01N 1/30; G01N 15/14; G01N 33/50

(52) U.S. Cl. CPC G01N 1/30; G01N 15/14; G01N 2015/1006; G01N 2001/302; G01N 2015/149; G01N 33/5097

(57) ABSTRACT

A method of sorting cells on a FACS includes providing a culture of cells stained with a dye. The dye is excited using photons at a first wavelength. A fluorescence and emission of the dye is collected at a second wavelength. Droplets of the cells are produced by pumping the cell culture at a first pressure through a nozzle having a nozzle diameter. The droplets are produced at a first frequency. The cells are sorted by a desired property. The desired property can include sorting the cells by size using at least one of a forward scatter area (FSC-A), a forward scatter height (FSC-H), a forwards scatter width (FSC-W), side scatter area (SSC-A), a side scatter height (SSC-H) and a side scatter width (SSC-W) of the fluorescence of the dye to determine a size of the cells.

### CROSS-REFERENCE TO RELATED APPLICATIONS

The present applications claims priority to and benefit of U.S. Provisional Application No. 62/137,093, filed Mar. 23, 2015 and entitled “Systems and Methods for Selecting Cellular Strains,” the entire disclosure of which is incorporated herein by reference.

### TECHNICAL FIELD

The present disclosure relates generally to systems and methods for selecting cell strains using staining and cell sorting.

### BACKGROUND

Algae, like higher plants, naturally produce an array of useful secondary compounds. Several current markets for algal biofuel products include the production of the antioxidant astaxanthin, omega oils for dietary supplements, and biofuels as a diesel substitute, while emerging markets include biopolymers for degradable plastics, supplements for aquaculture feed, and bio-fertilizer. In addition, algae can perform valuable services such as wastewater treatment and carbon sequestration. Specific strains of algae are used for specific products or services much the same way specific plant cultivars are suited to produce different products in different environments. Most food crops have typically undergone a long period of genetic improvement through domestication and more than 100 years modern breeding. However, most algal production systems are relatively new and they have not undergone the same genome-wide selection for advantageous traits.

Genetic improvement of algae has focused on manipulating traits through genetic engineering, where knowledge gained from genetic and biochemical studies are used to manipulate gene expression or introduce foreign genes that typically increase the output of a biosynthetic pathway. For example, in Chlamydomonas reinhardtii (C. reinhardtii), an increase in the expression of a thioesterase can alter the fatty acid profile while the transfer of the enzyme that regulates the carotenoid commitment step, phytoene synthase, increases carotenoid production. However, genetic reversion of such traits is a concern, as the relatively few genetic changes targeted can be lost in a culture environment in which engineered traits are not advantageous for survival. In addition, genetically engineered organisms face regulatory hurdles that typically cost tens of millions of dollars when bringing such products to market. In addition, public acceptance, especially in the nutraceutical industry, may also be an issue.

One viable approach for genetic improvement in algae is selection on populations, as microbes such as algae can evolve quickly in culture due to rapid cell division rates and the ability to culture large populations. Flow cytometry permits selection of microbes using fluorescent dyes to detect specific biosynthetic products such as oils and proteins. However, selection for quantitative increases in fluorescent staining can be confounded by variation in cell size, clumping behavior of cells, and uneven staining for desired traits.

### SUMMARY

Embodiments described herein relate generally to methods for selection of cells such as algal cells using staining and flow cytometry. Particularly, various embodiments described herein provide methods of live staining and dual staining of cells, and separating cells in a fluorescence assisted cell sorter (FACS) by size using a plurality of gates based on various fluorescence parameters of a staining dye used for staining the cells. Various embodiments also relate to separating astaxanthin rich cells from chlorophyll rich cells using a stain free method.

In some embodiments, a method for staining live cells includes growing cells to a stationary phase in a culture media. The cells are stained with a concentration of a staining solution which includes a dye dissolved in DMSO or other solvents, such as methanol or acetone. The DMSO has a concentration in the range of 0.5% to 2% by volume. Greater than 90% of stained cells remain alive after the staining.

In other embodiments, a method of dual staining cells for comparing a first culture of control cells and a second culture of experimental cells includes diluting the first culture of control cells to a first concentration. The second culture of experimental cells is diluted to a second concentration such that the second concentration is equal to the first concentration. The temperature of the first culture and the second culture is reduced to a first temperature. A volume of a first staining solution is added to one of the first culture and the second culture. The first staining solution includes a concentration of a first dye dissolved in a first concentration of DMSO. A second volume of water is added to one of the first culture and the second culture which does not include the first staining solution. The second volume is equal to the first volume. Each of the first culture and the second culture are incubated for a first time. The first culture and the second culture are mixed to form a mixed culture. A volume of a second staining solution is added to the mixed culture. The second staining solution includes a concentration of a second dye dissolved in a second concentration of DMSO. Greater than 90% of the experimental cells and control cells remain alive in the mixed culture after the dual staining.

In still other embodiments, a method of sorting cells on a FACS includes providing a culture of cells stained with a dye. The dye is excited using photons at a first wavelength. A fluorescence and emission of the dye is collected at a second wavelength. Droplets of the cells are produced by pumping the cell culture at a first pressure through a nozzle having a nozzle diameter. The droplets are produced at a first frequency. The cells are sorted by a desired property.

In some embodiments, a method for sorting astaxanthin rich cells from chlorophyll rich cells comprises providing a cell culture. A first portion of the cells included in the cell culture have a high concentration of astaxanthin relative to chlorophyll, and a second portion of the cells included in the cell culture have a high concentration of chlorophyll relative to astaxanthin. A laser having a laser wavelength that distinguishes between a chlorophyll fluorescence signal and an astaxanthin fluorescence signal is determined. The cells included in the cell culture are communicated through a fluorescence assisted cell sorter (FACS). The cells included in the cell culture are excited with the laser. The exciting causes each of the cells included in the cell culture to produce a fluorescence signal. The fluorescence signal comprises the chlorophyll fluorescence signal and the astaxanthin fluorescence signal. The fluorescence signal is passed through a first channel comprising a first band pass filter, and a second channel comprising a second band pass filter. A ratio of a first portion of the fluorescence signal of each cell that passed through the first channel to a second portion of the fluorescence signal that passed through the second channel is determined. In response to the ratio being higher than a predetermined threshold, it is determined that the corresponding cell has a high concentration of astaxanthin. The first portion of the cells having the high concentration of astaxanthin relative to chlorophyll is sorted from the second portion of the cells.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF DRAWINGS

The foregoing and other features of the present disclosure will become more fully apparent from the following description and appended claims, taken in conjunction with the accompanying drawings. Understanding that these drawings depict only several implementations in accordance with the disclosure and are therefore, not to be considered limiting of its scope, the disclosure will be described with additional specificity and detail through use of the accompanying drawings.

FIG. 1 is a schematic flow diagram of a method for staining cell such that greater than 90% of the cells remain alive, according to an embodiment.

FIG. 2 is a schematic flow diagram of another embodiment of a method for dual staining cells in a culture which includes a culture of experimental cells and a culture of control cells.

FIG. 3 is a schematic flow diagram of a still another embodiment of a method for sorting cells using a FACS.

FIG. 4 is an experimental work flow chart showing collection and control strategy. Two experimentally-evolved lines and one control line were derived from each of the wild-type strains for each starvation treatment. In total, 8 experimental lines and 4 control lines were produced. Control lines are sorted during each round from a population in the median, where a gate located at the median FITC level of cells was extended to capture 2.5% of cells.

FIG. 5 is a plot of distribution of stain intensity in three populations of algal cells. The populations represent C. reinhardtii grown to stationary stage and stained with BODIPY 505/515. The X axis (FL1) represents BODIPY emissions and the Y axis represents cell counts. The cumulative fluorescence of each population is shown in the dotted lines.

FIG. 6 panels A-D are plots of staining efficiency in relation to solvent concentration and cell density. Cells were stained with 800 picomoles of BODIPY 505/515 in 200 microliters of culture. Panels A-B show mean population emission of BODIPY fluorescence (FL1, 514/20) as a function of solvent concentration (panel A) and cell density (panel B). Panels C-D show staining variability as percent of unstained cells, which also represents cell death or toxicity, as a function of solvent concentration (panel C) and cell density (panel D). Each boxplot is constructed from three separate replicates.

FIG. 7 panels A-B are plots of dual population staining replicate data for a fluorometric readout of selective effects on the population. The plots show the BODIPY 505/515 (FL1, 514/20) and SYTO 84 (FL4, 586/15) fluorescence distributions for CC124 cells after one round of selection vs controls. The two panels represent a dye swap of the same populations, where the selected population was prestained with SYTO84 and the control population was mock stained (panel A) and vice versa (panel B), with the selected population showing early stage divergence with an extended subpopulation of high lipid accumulating cells (FL1, BODIPY) and an overall shift in the highest density of cells.

FIG. 8 panels A-D show sorting criteria that establish a narrow range of cell sizes and identify and exclude cell clumps. Panel A shows the forward scatter pulse width (FSC-W) and height (FSC-H) can be used as a readout of cell size, with graph showing scatter properties of 6 different bead sizes (2, 4, 8, 10 and 12 microns). Panel B shows C. reinhardtii cells stained for BODIPY sorted from forward scatter (FSC-W) intervals corresponding to beads of 10, 12, and 14 microns with cells sorted along with 14 microns beads shown in panel C, where cell clumps were observed. In panel D, the fluorescent intensity of cells collected in the intervals marked in B is measured for their actual cell diameter, as determined by microscopic image analysis. Vertical lines in panel D represent size distribution of cells sorted in FSC-W intervals marked, while horizontal lines represent beads of determined sizes and their actual forward scatter distribution.

FIG. 9 panels A-F show sorting protocols that selects cells in a narrow size range. Panels A-C represent different scatter properties used in successive AND gates by comparing panel A forward scatter area (FSC-A) vs. side scatter area (SSC-A); panel B forward scatter height (FSC-H) vs. forward scatter width (FSC-W); and panel C side scatter height (SSC-H) vs. side scatter width (SSC-W). Gates are drawn around density contours to exclude the 10% outliers in relation to center of mass in the density plot. In panel D, PerCP fluorescence is used as a proxy for chlorophyll content, which is another property for which the cells are filtered for uniformity. In panel E, FITC-A represents emissions for BODIPY staining, which is a quantitative readout for oil content. PerCP is shown again on the Y axis. The high and low gates represent the 2.5% most fluorescent cells and then the next quantile of 2.5% most fluorescent cells. Panel F shows the cells that fall into each gate and the surviving cells in each hierarchal filter.

FIG. 10 panels A-D show different C. reinhardtti strains responding differently to nitrogen starvation. In panel A, the response of different strains to either complete nitrogen starvation (N—) or a switch to Nitrate (NO₃⁺) as the sole nitrogen source. Y axis is accumulation of TAG fatty acids as measured by BODIPY staining on analyzed confocal images. Note CC124 (nit1/nit2) mutant cannot grow on NO₃ but its nitrogen starvation response is not as severe on NO₃ as it is in N—. Images display strain CC124 in nitrogen replete conditions (panel B), after 2 days of ammonia starvation in the presence of nitrate (panel C), and 2 days in N-free media (panel D).

FIG. 11 is a schematic flow diagram of an example method for sorting astaxanthin rich cells from chlorophyll rich cells.

FIG. 12 is a schematic block diagram of a computing device which may be used to perform operations of any of the methods described herein.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION OF VARIOUS EMBODIMENTS

Embodiments described herein relate generally to methods for selection of cells such as algal cells using staining and flow cytometry. Particularly, various embodiments described herein provide methods of live staining and dual staining of cells, and separating cells in a fluorescence assisted cell sorter (FACS) by size using a plurality of gates based on various fluorescence parameters of a staining dye used for staining the cells. Various embodiments also relate to separating astaxanthin rich cells from chlorophyll rich cells using a stain free method.

Embodiments of the staining and cell sorting methods for identifying and isolating cells described herein provide numerous benefits including, for example: (1) allowing staining of live population of cells (e.g., algae cells) such that substantially all of the cells remain alive; (2) allowing staining of a population of cells in a mixed culture of cells selectively with two dyes; (3) providing stringent selection of cells based on cell size using multiples gate which associate cell size with various fluorescent properties of a dye staining the cells; (4) allowing iterative selection of algal species based on genetic traits associated with lipid production; (5) providing a stain free method of separating astaxanthin rich cells from chlorophyll rich cells (6) improving bio fuel production by identifying algal species associated with highest lipid production.

Systems and methods described herein use fluorescence biochemistry combined with flow cytometry in order to apply highly specific selective pressures on algal populations for rapid domestication. A fluorescent dye is used to stain live cells for useful compounds, such as triacylglycerides used in biofuel production. With a fluorescent reporter for the trait of interest, the FACS rapidly screens millions of cells within minutes and isolates individual cells that express a higher level of fluorescence which corresponds to higher level of lipid production. Furthermore, the methods described herein establish cell-sorting criteria to minimize the effects of cell size and cell aggregates, provides a stringent readout of trait improvement over control populations, and also include selective pressures to maintain robust growth. The methods entail an iterative bottlenecking of the cell population, selecting rare natural variants and building up a series of genetic changes that improve harvest. While examples are shown primarily for cell selection based on fluorescence associated with lipid production, any other genetic trait can be used as the marker for separating and isolating cell providing optimal characteristics of the specific genetic trait. For example, the methods described herein can be used for selecting organisms based on genetic traits for improving production of omega oils, biopolymers, high protein feedstocks and/or other microbial products.

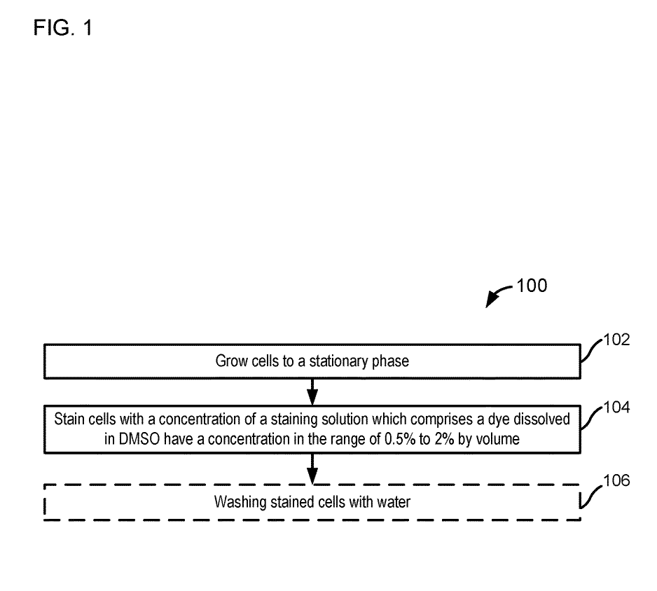

FIG. 1 is a schematic flow diagram of a method 100 for staining live cells with a dye. The method 100 can be used to stain any population of live cells, for example staining algae cells (e.g., C. reinhardtii cells) for lipid production.

The method 100 includes growing cells to a stationary phase in a culture media at 102. For example, algae cells can be cultured in any suitable culture medium (e.g., Tris-acetate-phosphate (TAP) medium, minimum essential medium (MEM), Modified Bold 3N medium (B3N), Blue Green No. 11, (BG-11), or COMBO medium). Cultures can be resuspended in variations of the culture media to induce macronutrients starvation such that the cells stop multiplying and enter the stationary phase. For example, the culture media can be nitrogen depleted.

The cells are stained with a concentration of a staining solution at 104. The staining solution includes a dye dissolved in DMSO (dimethylsulfoxide), methanol, or acetone having a concentration in the range of 0.5% to 2% by volume. For example, the DMSO can have a concentration of 0.5%, 0.6%, 0.7%, 0.8%, 0.9%, 1.0%, 1.1%, 1.2%, 1.3%, 1.4%, 1.5%, 1.6%, 1.7%, 1.8%, 1.9% and 2.0% by volume. In some embodiments, the concentration of DMSO is 1.0% by volume. In some embodiments, a concentration of the cells stained with the staining solution is in the range of 1×10⁶ cells/milliliter to 1×10⁸ cells/milliliter (e.g., 5×10⁷ cells/milliliter).

Any suitable dye can be used. In some embodiments, the dye includes BODIPY 505/515 which is formulated to stain lipids such triacylglycerides produce by cells. In other embodiments, the dye can include SYTO 84 which is an orange fluorescent nucleic acid stain. In still other embodiments, the dye can include SYTO® 9, CellTrace Violet, SYTO 85, ThiolTracker Violet, CellTracker Orange CMRA, Nile Red, Sudan Black B, VybrantDiO-C₁₈, C₁₁-BODIPY 581/591, BODIPY 493/505, β-BODIPY FL C5-HPA, Calcofluor White M2R, Wheat germ agglutinin-Texas Red®-X, any other dye or combination thereof.

The staining of cells does not have any substantial effect on the viability of the cells such that greater than 90% of stained cells remain alive after the staining. This is because the quantity of DMSO is sufficiently small to have minimal or no effect on the viability of the cells and majority of the cells in the cell culture remain alive. In some embodiments, the method 100 can also include washing the stained cells with water at 106. This can remove any residual DMSO remaining in the cell culture which can affect the viability of the cells.

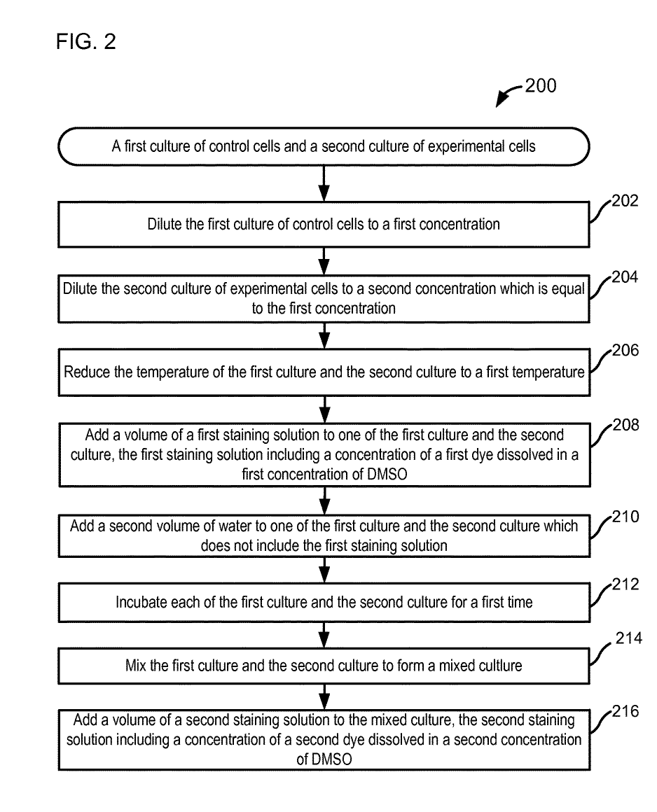

FIG. 2 is a schematic flow diagram of a method 200 of dual staining a population of cells in a mixed culture. The method 200 can be used with any cells (e.g., the algae C. reinhardtii) and can be used for comparing a first culture of control cells and a second culture of experimental cells included in a mixed culture. In some embodiments, the method 200 can be used to dual stain cells to enable determination of a ratio of neutral lipids between cells subjected to stringent selection (extreme 2.5% quantile), lower selection (second 2.5% quantile) and a median selection (median 2.5% quantile of control cell population).

The method 200 includes diluting a first culture of control cells to a first concentration at 202. A second culture of experimental cells is diluted to a second concentration at 204, such that the second concentration is equal to the first concentration. Each of the first culture and the second culture can include cells recovered from cryopreservation or cultures from cell lines that have a minimum of 2.0×10⁷ cells left over after cells have been isolated for sorting. In some embodiments, each of the first concentration of the first culture and the second concentration of the second culture includes 5.0×10⁶ cells/milliliter. The cells can be diluted using water or a culture medium (e.g., any of the culture mediums described herein).

The temperature of the first culture and the second culture is reduced to a first temperature at 206. In some embodiments, the first temperature is less than 15 degrees Celsius (e.g., 14 degrees Celsius, 13 degrees Celsius, 12 degrees Celsius, 11 degrees Celsius or 10 degrees Celsius). The reducing of the temperature of the cells to the first temperature can aid the cells in better surviving the operations of method 200. For example, in some embodiments, the cells are centrifuged, for example to isolate and resuspend cells and the reduced temperature can help the cells to survive the centrifuging.

A volume of a first staining solution is added to one of the first culture and the second culture at 208. The first staining solution includes a concentration of a first dye dissolved in a first concentration of DMSO. In other words, either the first culture or the second culture is stained with the first dye but not both. In some embodiments, the first dye included in the first staining solution is SYTO 84. In some embodiments, the first concentration of DMSO is in the range of 0.5% to 1% by volume (e.g., about 0.5% by volume).

A second volume of water is added to one of the first culture and the second culture which does not include the first staining solution at 210, such that the second volume is equal to the first volume. That is the water is added to the other culture of cells which was not stained with the first dye. The water serves as a mock stain so that the volume of the first culture and the second culture remains the same, ensuring that conditions of the first culture and the second culture are exactly the same.

Each of the first culture and the second culture is incubated for a first time at 212. For example, the first culture and the second culture can be incubated for 10 minutes to allow the first dye to stain either the first culture or the second culture to which the first staining solution was added. In various embodiments, the cells can be subjected to shaking (e.g., on a rotary shaker at an rpm in the range of 500 rpm to 1,000 rpm) during the incubation. The shaking can keep the cells homogenous, promote the staining and prevent the cells from forming clumps.

The first culture and the second culture are mixed to form a mixed culture at 214. A volume of a second staining solution is added to the mixed culture. The second staining solution including a concentration of a second dye dissolved in a second concentration of DMSO. In some embodiments, the second dye can include BODIPY 505/515. In such embodiments, the control cells and the experimental cells can include lipid producing algae. Furthermore, the second concentration of the DMSO can be equal to the first concentration of DMSO (e.g., in the range of 0.5% to 1% by volume). In some embodiments, before adding the second staining solution to the mixed culture, the mixed culture is subjected to repeated washes to reduce the concentration of the DMSO in the mixed culture to less than 0.001% (e.g., about 0.007%).

In this manner, one of the experimental cells or the control cells included in the mixed culture are stained with the first dye and the second dye, while the remaining cells are only stained with the first dye. Dual staining of the cells using the method 200 can be performed on live cells and does not affect or minimally impacts the viability of the cells. For example, greater than 90% of the experimental cells and control cells can remain alive in the mixed culture after the dual staining.

In some embodiments, the dual staining process of method 200 can be used to study multiple traits of the cells within the same culture. For example, in some embodiments, the first dye can be SYTO 84 which is used to monitor genetic traits of the cells by staining the nucleic acids of the cells. The second dye can include BODIPY 505/515 for staining lipids produced by lipid producing algae cells. The cells in the mixed culture stained with both the stains provide information on changes in genetic makeup via fluorescence of the SYTO 84, and associated changes in lipid production via fluorescence of the BODIPY 505/515. The cells in the mixed culture stained only with the BODIPY 505/515 provide information solely on lipid production and can be used for comparison or for data normalization. In this manner, dual staining of cells using method 200 can allow identification of algae cells having genetic traits providing optimum lipid output. The identified cells can be isolated and used as a superior source of lipid (e.g., biofuel) production.

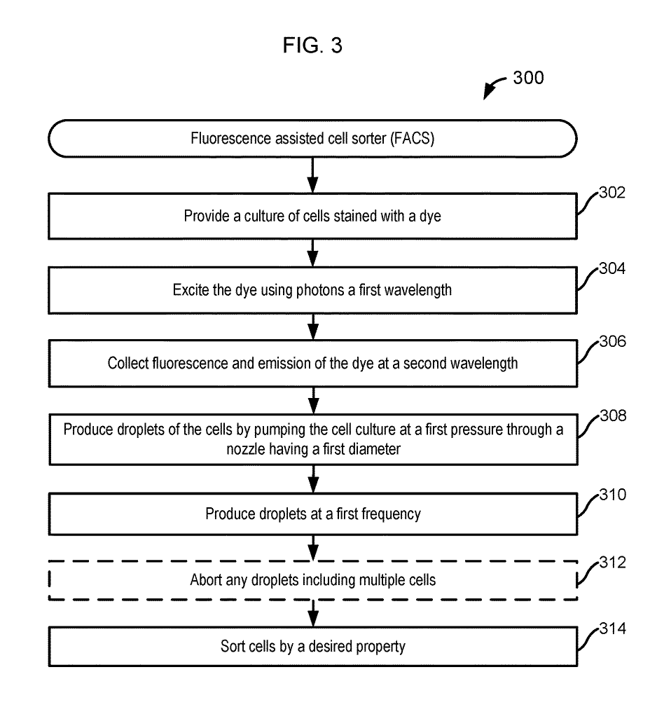

FIG. 3 is a schematic flow diagram of a method 300 of sorting cells on a FACS. For example, the method can be used to sort cells (e.g., algae cells) based on genetic traits of the cells. Such cells can be dually stained, for example using the method 200 before sorting the FACS.

The method 300 includes providing a culture of cells stained with a dye at 302. In some embodiments, the cells include a homogenous culture of experimental cells. The cells can be stained with BODIPY 505/515. In other embodiments, the cells can include a mixed culture of experimental cells and control cells. At least a portion of the cells included in the mixed culture (either the experimental cells or the control cells) can be dually stained with a BODIPY 505/514 and SYTO 84, while the remaining cells are stained with SYTO 84 only. The dual staining can be performed, for example using the method 200 or any other suitable methods described herein.

The dye is excited using photons at a first wavelength at 304. In some embodiments, the dye is BODIPY 505/515. In such embodiments, the first wavelength can be 488 nm, for example produced using a blue laser. A fluorescence and emission of the dye is collected at a second wavelength at 306. In embodiments in which the dye is BODIPY 505/515, the second wavelength is in the range of 505 nm to 515 nm. For example, the fluorescence can be collected at 505 nm and the emission can be collected at 515 nm. In further embodiments, autofluorescence of the cells can also be measured between a wavelength of 685 nm to 735 nm. In various embodiments, a window gate extension of the FACS is set to 0. In still other embodiments, a threshold of the forward scattered light is set to be less than 8,000.

Droplets of the cells are generated by pumping the cell culture at a first pressure through a nozzle having a nozzle diameter at 308. In some embodiments, the nozzle diameter of the nozzle is about 70 microns. In other embodiments, the first pressure 70 psi. The size of the nozzle and/or the pressure can be varied to control a droplet size such that each droplet includes a single cell.

The droplets are generated at a first frequency at 310. In some embodiments, the first frequency is about 7,500 Hz. In particular embodiments, any droplet which includes multiple cells are aborted at 312. For example, a sorting mask can be used to ensure that only a droplet containing multiple cells is aborted but not the subsequent drop.

The cells are sorted by a desired property at 314. In some embodiments, sorting the cells by a desired property includes sorting the cells by size using at least one of a forward scatter area (FSC-A), a forward scatter height (FSC-H), a forwards scatter width (FSC-W), side scatter area (SSC-A), a side scatter height (SSC-H) and a side scatter width (SSC-W) of the fluorescence of the dye (e.g., BODIPY 505/515) to determine a size of the cells.

In some embodiments, the FSC-W and FSC-H can be used as a readout of cell size for cell sorting. The difference between the size of the sorted cells can be about 2 microns. For example, cells having a diameter of 2 microns, 4 microns, 6 microns, 8 microns, 10 microns and 12 microns can be sorted using the FSC-W and FSC-H as a proxy for cell size. For example, scatter plots of the FSC-W and FSC-H of the fluorescence of the dye can be drawn and used to sort the cells by size.

In some embodiments, the cells are sorted by size using a first gate, a second gate and a third gate. In such embodiments, the first gate sorts the cells by comparing the FSC-A versus the SSC-A, the second gate sorts the cells by comparing the FSC-H versus the FSC-W, and the third gate sorts the cells by comparing the SSC-H versus the SSC-W. The gates can be nested, for example the cells can be sorted by the first gate, followed by the second gate and the third gate. Furthermore, each gate can be drawn based on 100,000 events recorded immediately after the mean fluorescence has peaked for the cells.

In particular embodiments, the first gate sorts for a first portion of the cells which includes a top 2.5% of the cells based on a rank of their fluorescent intensity. The second gate sorts for a second portion of the cells which includes 2.5% of the cells subsequent to the first portion based on fluorescent intensity, and the third gate sorts for a third portion of the cells which includes an interval containing 2.5% of the cells whose fluorescent intensity is centered on the mean fluorescence level of all cells. The third gate captures the control population.

Each of the first gate, the second gate and the third gate can include density plots of the two fluorescence parameters being compared in each gate. Each gate is drawn to align with software generated contours to reject the most extreme 5-10% of the hits per plot (e.g., the 10% outliers). Moreover, the gates can be drawn for each cell sample to contain a proportion of an initial number of events (e.g., 100,000 events) that fall within the 95-97.5^{th} percent extremes and also the 97.5^{th} percentile.

In some embodiments, the cells include algal cells. Algae cells include chlorophyll to perform photosynthesis. In such embodiments, the cells are also sorted by chlorophyll content using a fourth gate. The fourth gate sorts cells by comparing a PerCP-Cy5.5 height (PerCP-H) versus a PerCP-Cy5.5 width (PerCP-W). The PerCP can serve as a proxy for the chlorophyll content of the cells which can allow filtering of the cells based on uniformity. In other organisms like bacteria or yeast, a TRITC emissions channel can be used to measure autofluorescence arising from innate cellular properties.

### EXPERIMENTAL EXAMPLES

The methods described herein were used on to stain and sort the model algal system, C. reinhardtii, using fluorescent stains for fatty acids to increase triacylglceride production over seven selective cycles (rounds). The protocol below serves to show how a useful product (in this case oils for biofuel production) can be dramatically improved using this accelerated domestication process, which takes only a few weeks.

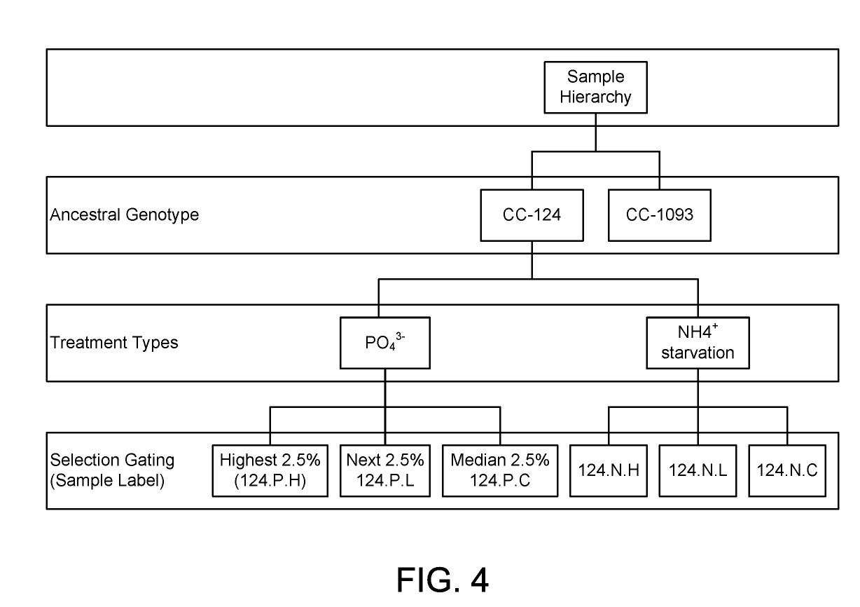

The experiments were designed to address several potential problems that arise in algal evolution experiments, including: 1) not all strains respond equally to selection pressures; 2) the same strain may respond in a different way in repeat experiments; 3) different levels of selective pressure can yield different types of genetic trait evolution, where extreme pressures may or may not select on artifactual cell properties. Experiments were conducted on two different lines from the same species. FIG. 4 shows two experimentally-evolved lines and one control line derived from each of the wild-type strains for each starvation treatment. In total, 8 experimental lines and 4 control lines were produced

All samples were replicated including controls, in order to gain confidence that selective pressures were effective on the different stains. To ensure homogeneity control cells were grown, centrifuged and sorted along with the selected population of cells, where controls were stained and selection gates were placed at the median fluorescent level (i.e., selecting for no net change in BODIPY staining). This ensures that improvements in BODIPY staining are due to the quantitative selection criteria and not an artifact of the protocol itself.

Furthermore, the sorting method gate on two selected populations, one capturing the top 2.5% BODIPY fluorescent cells and a second gate capturing the next 2.5 percentile of BODIPY stained cells. This guards against cases in which extreme outliers are artifacts of staining irregularities. Finally, all sorted populations, including controls, represent 2.5 percent intervals of the total population. This permits efficient collection of equal numbers of cells from each population and controlled for bottleneck size.

The number of cells interrogated in the flow stream of the FACS represents the effective population size for the sake of selective pressure. The combination of cell sorting time and organism mutation rate were coordinated to result in the interrogation of a sufficient number of variants in any given round of selection. For C. reinhardtii, mutation rates vary from 0.038 to 0.0082 mutations/genome/generation i.e. one mutant per 26 to 122 daughter cells. In order to prevent electronic aborts and multiple cells per droplet, a target event rate at 7,500 Hz and a collection speed of 100 Hz were selected.

Given mutation rates, sorting parameters, and expected rate at which mutations create non-synonymous changes in the coding gene space, phenotypes of between 61 to 288 mutant genomes/second were interrogated. The experiments were designed to interrogate about 10 potentially function-altering mutations per gene in each round of selection to provide thorough coverage for loss-of-function variants and potentially allow interrogation of some neofunctional variants. Under the low mutation rates of C. reinhardtii, 1.5×10⁷ cells were sorted which was accomplished in about 30 minutes. To avoid genetic drift and account for resource constraints, a bottleneck size of 200,000 cells per independent line was set.

C. reinhardtii cultures were averaged for total cell count of 8.6×10⁷ for N— and 1.8×10⁸ for PO₄³⁻ before screening. The growth protocols to prepare cultures for FACS screen in fatty acid accumulation experiment were configured to evolve a strain that accumulates fatty acids more quickly and to higher amounts than controls during a nutrient starvation.

Strains CC-1690 wild type mt+[Sager 21 gr] and CC-124 wild type mt− [137c, carrying the nit1 and nit2 mutation] were obtained from the Chlamydomonas Resource Center at the University of Minnesota. CC-1093 wild type mt− [137c, UTEX 2247, also carrying the nit1/nit2 mutation] were obtained from the University of Texas Culture Collection. Haploid cells were propagated asexually in liquid culture using Tris-Acetate-Phosphate (TAP) media with a modified trace metal solution. Cultures were resuspended in variations of TAP to induce macronutrient starvation. These include omitting either the phosphate buffer for P-TAP or NH₄Cl for N-TAP.

An alternative nitrogen source formulation (NO₃ TAP), supplemented with 3.5 millimolar NaNO₃ was also used. For the growth phase, cultures were shaken at 150 rpm on a rotary shaker under 90 micromole photons/m² s provided by cool white fluorescent bulbs in a growth chamber synchronized to a 12:12 light/dark cycle at 28 degrees Celsius. Cells were harvested for resuspension, washing, and staining by centrifugation. Volumes greater than 1.5 milliliter were diluted with water to either 10 or 40 milliliter in 14 or 50 milliliter centrifuge tubes. Cells were pelleted in a swinging bucket rotor was spun at 800 g for 10 minutes at 10 degrees Celsius.

All lines were inoculated simultaneously in 3 milliliter volume in 12 well plates. Disposable 1 milliliter pipette tips were used to transfer a small mass of scraped cells from the agar slants on which they were received. Culture volume was doubled with fresh media after 48 hours and 0.5 milliliter of each culture was sampled for counting. If necessary, cultures were diluted at 72 hours with additional media to 1.0×10⁶ cells/milliliter to avoid early starvation. At 96 hours, the cells were washed and each culture was resuspended in 1 milliliter, and one third was aliquoted into 10 milliliter fresh media and cryopreserved on day 5. The remaining fraction was resuspended at a fixed density of 1.5×10⁵ cells/milliliter, which used 25±5 milliliter of the appropriate starvation media.

The solution was put in a glass Erlenmeyer flask topped with a polypropylene cap. Both vessel types were sealed with 1-inch wide hypoallergenic paper tape (Kendall). After 3 days of starvation, cells were washed and resuspended in TRIS-buffered saline (TBS) and held at 10 degrees Celsius for 2 to 3 hours for FACS. The cells were sorted into 2 milliliter TAP, and immediately transferred back to the initial condition of 3 milliliter in a 12-well microplate. The only difference in procedure between treatment types was that nitrogen starved cells required an additional day of recovery before measurable growth was observed.

### Cell Staining

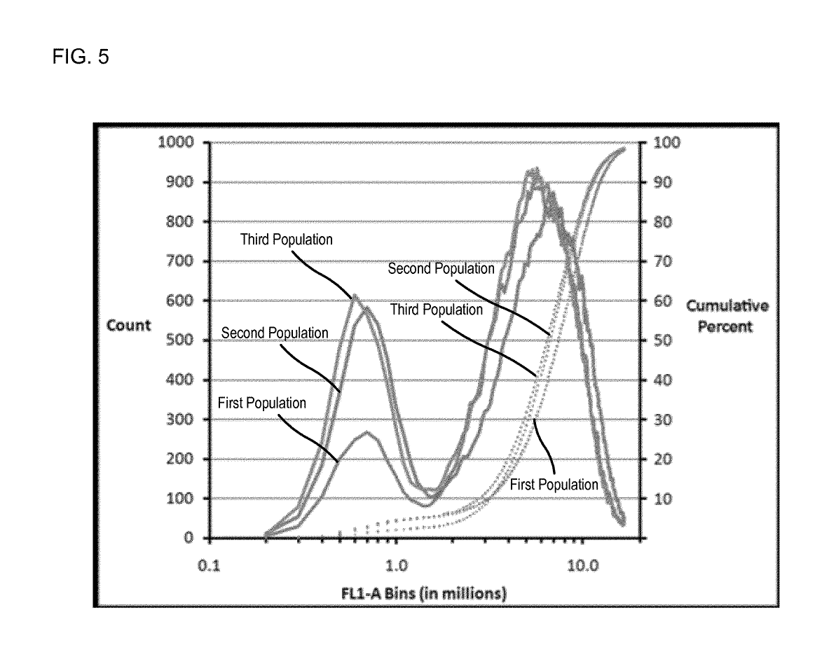

Variability in staining can lead to false positives and false negatives in selection. The staining protocol of method 100 was used and a solvent concentration, incubation time and cell density to ensure cells remain alive was determined. In some instances, live staining can yield is a bimodal distribution of staining intensities indicating that staining was not uniform and/or staining conditions may be lethal to a subpopulation of cells. FIG. 5 shows distribution of stain intensity in three populations of C. reinhardtii. The populations represent C. reinhardtii grown to stationary stage and stained with BODIPY 505/515. The X axis (FL1) represents BODIPY 505/515 emissions and the Y axis represents cell counts. The cumulative fluorescence of each population is shown in the dotted lines.

To test solvent effects, cells grown in nitrogen replete conditions to stationary phase (where a moderate amount of lipids are present) were stained with BODIPY 505/515 (5 microgram/milliliter) in varied DMSO concentrations. The resultant distribution was evaluated for 150,000 cells by flow cytometry in triplicate using a BD Accuri C6. Both CC-1093 and CC-124 were tested. First, it was determined that 0.5 to 1% by volume of DMSO was a minimal solvent concentration for sufficient stain intensity. Increasing DMSO concentration in range of 1 to 4.8% led to modest increases in mean fluorescence but more dramatically increases the percent of unstained cells, indicating that the higher concentrations of DMSO is increasing cell death. Given the modest gains in fluorescent intensity and the dramatic increases in cell death, the DMSO concentration was kept at 1%.

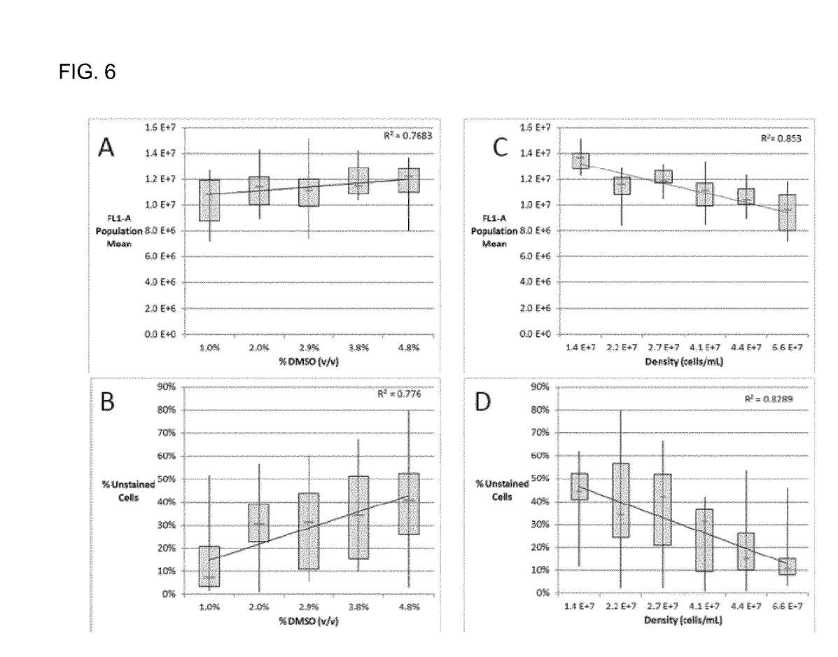

FIG. 6 panels A-D show staining efficiency in relation to DMSO concentration and cell density. Cells were stained with 800 picomoles of BODIPY 505/515 in 200 microliters of culture. Panels A-B show mean population emission of BODIPY fluorescence (FL1, 514/20) as a function of solvent concentration (panel A) and cell density (panel B). Panels C-D show staining variability as percent of unstained cells, which also represents cell death or toxicity, as a function of solvent concentration (panel C) and cell density (panel D). Each boxplot is constructed from three separate replicates. The number of cells in the 0.2 milliliter staining solution was varied from 4.0×10⁶ to 1.5×10⁷ cells. Increasing densities led to modest losses of overall BODIPY staining intensity (FIG. 6 panel C) but also appeared to lower cell death during staining (FIG. 6 panel D), as indicated by fewer unstained cells. The higher cell densities appeared optimal as staining levels were high enough to detect and minimal cell death occurred.

The same staining method was performed using a second dye SYTO 84. An extra wash step was performed after staining which can reduce staining variability, for example by reducing cell death from exposure to solvent. Based on this, for single dye staining three tubes, each containing 2 milliliter of culture volume, are pelleted at 600 g for 1 minute and 800 microliters is removed. After the pellet is gently loosened by hand shaking, 1.5 microliter of 200 micromolar BODIPY 505/515 is added to each tube. After adding the stain, each tube is immediately vortexed at the lowest speed and all three are incubated for 5 minutes. The cells are then washed with 1 milliliter H₂O and diluted with 1 milliliter sheath fluid for cytometric analysis. Triplicate measurements of the mean FL1-A channel are recorded.

Cells were dual stained using a variation of the method 200. The method provides a stringent, high throughput quality control during intermediate or final rounds of selection or can be used during selection rounds to establish gates based on cells that represent extremes of the control population or exceed controls in later rounds of selection. In this method, either an experimental culture is marked with a tracker dye and then mixed with the control or vice-versa. This marks either the control population or the treated population beforehand, permitting both populations to be stained simultaneously with the dye of interest. By staining simultaneously and recording the distributions simultaneously, inherent batch variability is avoided in staining brightness of vital dyes, such as BODIPY 505/515. The density of cells, staining culture volume, stain volume, and resuspension density (at the start of starvation) is held exactly equal for all cultures stained in both methods to enhance validity of the comparison.

For dual stain analysis, a pair of 2 milliliter samples are taken from the diluted experimental and control cultures. The 4 tubes are each reduced in volume to 0.2 milliliter and 1 microliter of 400 micromolar SYTO 84 is added to one of each pair (marked) and a matching volume of water is added to the other (unmarked). Both are left to incubate for 10 min, shaking at 850 rpm. The cells are then washed twice with 1 milliliter of water and brought back up to volume. Table I shows volumes added at each step and working DMSO concentration.

TABLE I
Volumes of DMSO, staining culture, and percent ratio
over the course of dual staining.

| | Culture volume (μL) | DMSO (v/v) |
|---|---|---|
| Culture is sampled | 2000 | 0 |
| Spin 1 | 200 | 0 |
| SYTO 84 added | 201 | 0.5% |
| Wash 1 | 1201 | 8.33E−04 |
| Spin 2 | 201 | 8.33E−04 |
| Wash 2 | 1201 | 1.39E−04 |
| Spin 3 | 201 | 1.39E−04 |
| Original volume/density | 2000 | 1.04E−05 |
| Halve | 1000 | 1.40E−05 |
| Mix w/control | 2000 | 7.00E−06 |
| Spin 4 | 200 | 7.00E−06 |
| Add BODIPY 505/515 | 201.5 | 0.75% |
| Wash 1 | 1201.5 | 0.12% |
| Spin 5 | 201.5 | 0.12% |
| Dilute with Sheath | 1201.5 | 2.1e−04 |

For every pair wise comparison, dye swaps are performed such that experimental cells and control cells are alternatively marked with SYTO 84. After SYTO 84 staining, the experimental cells and control cells are mixed and stained together for the selection dye, in this case, BODIPY 505/515. The BODIPY 505/515 staining is performed as detailed above with one cell population.

In dual staining, the concentration of DMSO was kept at 0.75% by volume as cells are exposed to DMSO multiple times. Furthermore, the DMSO is reduced to 0.0007% before the second stain is introduced. Repeated washing and pelleting is performed to reduce the volume of stain remaining in solution by nearly 3 orders of magnitude before the unmarked population is introduced (to minimize carry over staining). Table II shows a control population of cells grown to stationary phase and split into two aliquots.

TABLE II
Effect of mixing 100 microliter of unstained cells at
the same density to 100 microliters of washed cells

| | % | Cell | Mean | CV | Mean | CV |
|---|---|---|---|---|---|---|
| Group | DMSO | Density | 586/15 | 586/15 | 514/20 | 514/20 |
| Marked | 1.5% | 1.38E+07 | 1,673,224 | 48% | 4,476,526 | 37% |
| Un- | 0% | | 176,672 | 34% | 5,029,365 | 34% |
| marked | | | | | | |

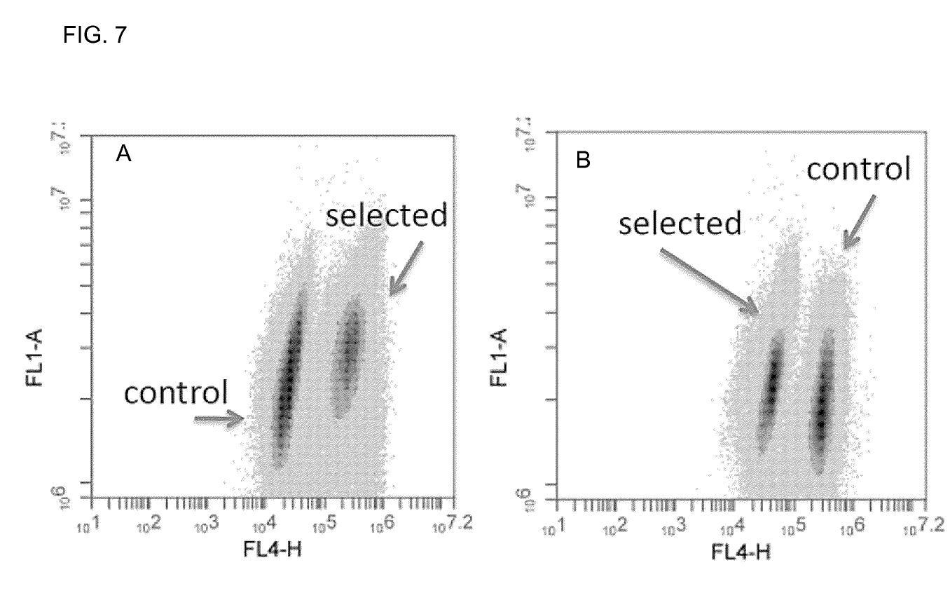

Half the aliquot was stained with SYTO 84 and the other half was mock stained. Both aliquots were then combined and stained with BODIPY 505/515. The stained half shows an order of magnitude more signal in the SYTO 84 channel (586/20 nm), while both aliquots show about the same intensity in the BODIPY channel (514/20). Effective separation of marked cultures is achieved when the average SYTO 84 signal is >8× higher than the unmarked cells as shown in FIG. 7 panels A-B.

The dual staining method was also tested on experimental vs. control cultures. A nitrogen starved culture was selected for variants expressing higher levels of BODIPY in one round. The control population came from the same batch of cells but were sorted from the median 2.5%. Cells from the two sorts were regrown, subjected to the dual staining protocol, and analyzed by flow cytometry in the SYTO 84 and BODIPY 505/515 channels (FIG. 7 panels A-B). The results show that the selected cells extends further into the BODIPY 505/515 channel in both outcomes of the dye swap experiment (FIG. 7 panel B). Furthermore, dual staining method also permits a quantitative comparison of the distribution of the two cell populations. For example, the most dense region of the treated cells also shows a shift toward higher BODIPY staining.

### FACS Setup and Cell Sorting

Cell sorting was performed using a BD FACSAria II with Aerosol Management. The FACS flow path was flushed with ethanol for 10 minutes and ultrapure water for 30 minutes before sorting. To avoid potential cross contamination, a solution of 2% sodium hypochlorite followed by ultrapure water were passed through a flow path of the FACS for 1 minute each between each independent line. Autoclaved TRIS-buffered saline was used for sheath fluid. The blue laser (488 nm) was used to excite BODIPY 505/515 fluorescence and emissions were collected between 505-545 nm. Autofluoresence was measured between 685-735 nm. The window gate extension was set to 0.5 and threshold to FSC<8,000. A custom sorting mask was created to ensure that only a droplet containing a conflict (i.e. multiple particles) was aborted but not the subsequent drop. The drop delay was calculated using the Accudrop Beads (BD) before every sort and any time the stream needed restarting. A 70 micron nozzle was used and the default sheath pressure of 70 psi was used. The flow rate was varied between tubes to maintain an event rate between 7,500±1,000 Hz.

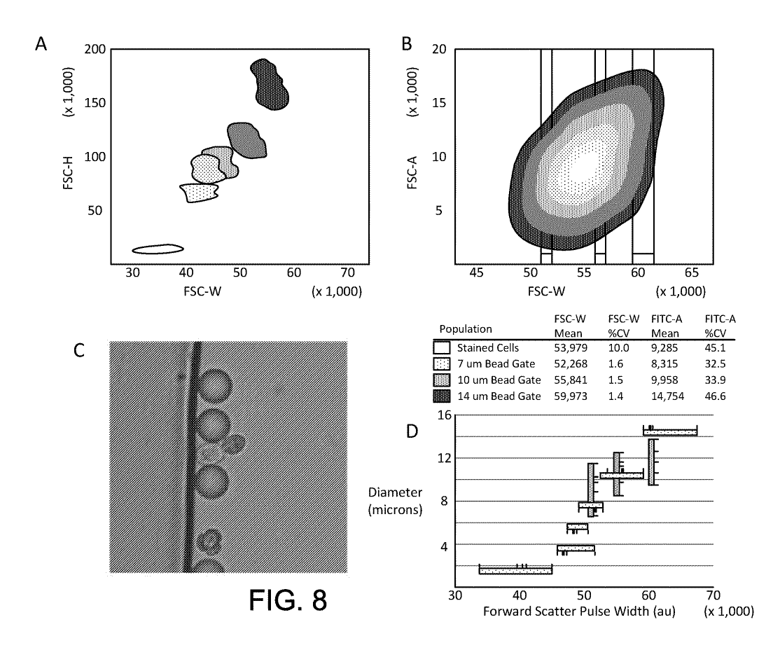

Stringent calibration for particle size detection is needed for subsequent steps. PMTs were adjusted such that all cells were recorded in the region of highest resolution i.e. 103 to 105 for logarithmic (fluorescence) data and 1.0×105 to 2.0×105 for linear (light scattering) data. Calibration for area scaling factor was performed for each laser. The Polystyrene Particle Size Standard Kit (Spherotech Inc) was used to record mean SSC-W and FSC-W values for particles of known diameter. These averages were plotted against bead diameters and a nonlinear calibration curve was fitted (FIG. 8 panel A).

### Gating Strategy

The FACS was calibrated to standard size particles, to determine the range in which single cells of relatively uniform size were most likely present (FIG. 8 panel A). To test scatter properties as a proxy for cell size on real cells, beads and cells are mixed and the cell mixture was sorted based on forward scatter properties that capture specific cell and bead sizes (FIG. 8 panels B-C). The plot in FIG. 8 panel D shows the ability to capture cells in specific 2 micron increments. The variation in cell size when selecting from a narrow forward scatter window can be observed in FIG. 8 panels B and D). The method can be used to observe the forward scatter range that provides the least variation in cell size. In addition, it can be used to observe the size range that begins to include clumps of cells, such as the clumps observed in the experiment when sorting the C. reinhardtii cells from region of 12 micron beads (FIG. 8 panel C).

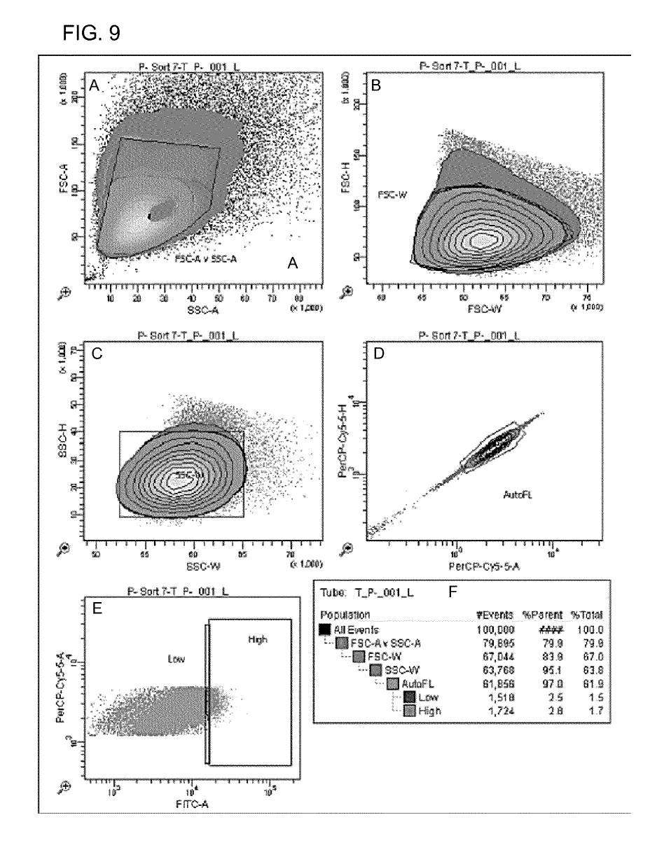

Using these size standards as a reference, a nested series of purification gates were drawn on two dimensional plots displaying the distribution of fluorescence pulse widths (W), areas (A), and heights (H) in the forward scatter (FSC), side scatter (SSC), and autofluoresence (PerCP-Cy5.5) channels (FIG. 9 panels A-C). Gates were drawn to align with software-generated contours to reject the most extreme 5-10% of hits per plot. The gates were nested in the following order gate 1: FSC-A vs. SSC-A; gate 2: FSC-H vs. FSC-W; gate 3: SSC-H vs. SSC-W; and gate 4: PerCP-Cy5.5-H vs. PerCP-Cy5.5-A (FIG. 9 panels A-C).

The PerCP gate was used as a proxy for chlorophyll content and outliers were also removed using upper and lower thresholds on this channel (FIG. 9 panel D). Cells from each independent line were split across three tubes for staining and sorting. It was observed that a FITC-A population mean would increase significantly and steadily during the first 1 to 2 minutes of sample analysis, regardless of stain incubation time. After peaking, the mean would slowly decrease by >1%. Therefore, gates were drawn based on 100,000 events recorded immediately after the mean had peaked for each tube and usually between 10 to 30% were gated out.

The fluorescence pulse height (FITC-H) and area (FITC-A) emitted by BODIPY 505/515 was used as a final selection threshold on the trait of interest. The gates were drawn for each sample to contain the proportion of the initial 100,000 events that fell within the 95-97.5^{th} percent extremes (lower selection) and also the 97.5^{th} percentile (stringent selection, FIG. 9 panel (E)). While only one was actively used to collect at a time, both were dragged horizontally and leftward in real time over the course of the 10 minutes of processing required per tube. The screen was set to update every 10,000 events and the gates were moved horizontally leftward to include the same proportion of the population as the signal amplitude diminished.

### Nitrogen Response and Assimilation Pathway

The methods described herein were used to monitor nitrogen response and assimilation pathway by targeting novel components of the signal transduction pathway. BODIPY 505/515 accumulation was used to investigate the fatty-acid accumulation response of cells to nitrogen in different genetic backgrounds. Experiments were performed based on the methods described herein using starvation stress to induce fatty acid accumulation in C. reinhardtii cells.

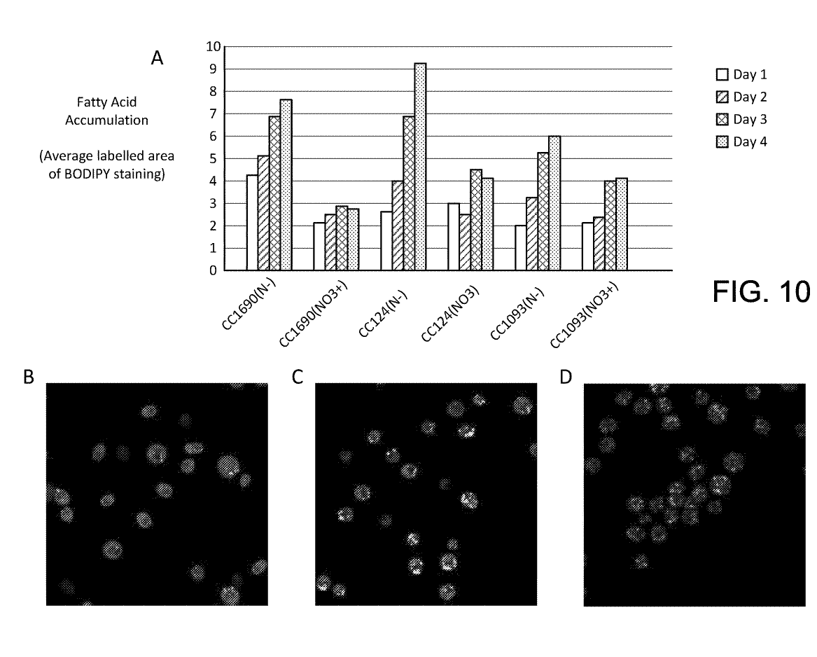

The different strains were first tested for their response to nitrogen starvation (FIG. 10 panels A-D). The wild type strain (CC1690) showed a typical response to nitrogen starvation, where BODIPY 505/515 staining showed fatty acid accumulation over four days of nitrogen starvation (N—). The strain shows almost no response when it is switched from its ammonia-based media (TAP) to a custom TAP media with an alternate source of nitrogen (inorganic nitrogen, NO₃), showing that the cells sensed the inorganic nitrogen and did not go into starvation response.

The nit1/nit2 mutant strain (CC124), which cannot assimilate nitrate, grows normally on the ammonia-based media and, just as does wild type; it increases fatty acid accumulation in nitrogen-free media (N—). When CC124 is switched to a nitrate-only media (NO₃), cells no longer grow, showing it cannot assimilate the NO₃. However, CC124 shows an attenuated starvation response in NO₃-only media, much closer to wild type than expected for a complete block in nitrate signaling. Similarly, CC1093, which also carries multiple mutations in the nitrate assimilation pathway, also shows an attenuated response, i.e., closer to wild type than its response on N-media.

The result indicates that, although the nitrate assimilation mutants cannot utilize NO₃, they still appear to sense the nitrate in the media and dampen their starvation response. These results provided the basis for an “enhancer” evolution screen to increase the starvation response of CC124 in the presence of nitrate, where such a screen should target as yet unknown signaling components in the nitrate-sensing pathway.

For CC124 and CC1093 strains, cells were grown on TAP media and after the culture reached the appropriate size, spun down, and transferred to NO₃ media. Cells were allowed to starve for three days and then the highest BODIPY 505/515 staining cells were selected according to the protocol detailed above. For an alternative target evolution experiment, phosphorous starvation was induced by switching cells to a phosphorous free TAP media. Similarly, cells were selected for higher BODIPY 505/515 staining after two days of phosphorous starvation.

The four selection experiments, with controls and replicates, were carried out over seven rounds of selection. The results after the 7 rounds, assessed by multiple single and dual staining analyses, are shown in Table III, where replicates are averaged together.

TABLE III
Estimated improvement in lipid productivity after gate based selection of species.

| | | Fluorescence Data | | | | Estimated | |
|---|---|---|---|---|---|---|---|
| | | (% of control | | Estimated | | Productivity | |
| | | pop. Mean) | | [TAG] (mg/L) | | (mg/L/day) | |
| | Starvation | High Gate | Low Gate | High Gate | Low Gate | High Gate | Low Gate |
| Strain | Type | Group | Group | Group | Group | Group | Group |
| cc124 | P- | 1.92 | 1.41 | 580.8 | 426.5 | 82.9 | 60.8 |
| cc1093 | P- | 2.43 | 1.4 | 735.1 | 423.5 | 104.9 | 60.4 |
| cc124 | N- | 1.02 | 1.49 | 381.2 | 517.3 | 54.4 | 73.8 |
| cc1093 | N- | 1.11 | 1.09 | 335.8 | 329.7 | 47.9 | 47.0 |

The most dramatic gains were in the phosphorous starvation regime, in which CC1093 increased in fatty acid production over control by 2.4 fold. CC124 showed a similar high increase after selection in the phosphorous starved environment (1.92 fold increase over control). In both cases, the most stringent gates (highest selective criteria) led to the most dramatic gains for phosphorous starvation. For nitrogen starvation in NO₃, only CC124 showed an improvement (about 1.5 fold) with the low gates. This was reasonable since the primary targets of the selection experiment (nitrate assimilation genes found in prior screens) were already mutant. This evolution approach can target redundant members of a gene family that show incremental changes alone over successive rounds. In addition, the large saturation of mutations screened could possibly also uncover gain of function variants in the population that have arisen by chance to increase fatty acid accumulation.

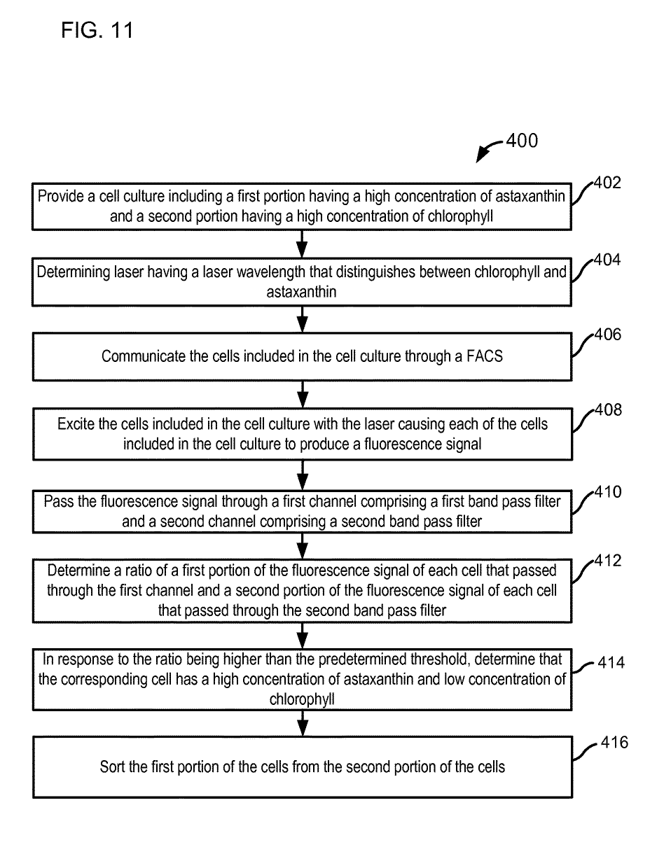

FIG. 11 is a schematic flow diagram of an example method 400 for sorting cells based upon certain chemical compositions within the cell that exhibit different fluorescence when using an optimized light source, as one example the method 400 can be used to differentiate astaxanthin rich cells and chlorophyll rich cells. Such cells may include, for example Haematococcus pluvialis (H. pluvialis), a commercially valuable algal species that is used to produce the nutraceutical product astaxanthin, a powerful antioxidant carotenoid. Expanding further, H. pluvialis is a green algae that will rapidly produce astaxanthin in response to stress such as nitrogen starvation and high light exposure. The process involves a transition from green to red appearance that marks a transition from chlorophyll-rich (i.e., cells having a high concentration of chlorophyll relative to astaxanthin) to astaxanthin-rich individual cells (i.e., cells having a high concentration of astaxanthin relative to chlorophyll). However, under fluorescence detection, astaxanthin and chlorophyll have a highly similar emission spectrum, making them difficult to distinguish. The inability to distinguish the two spectra may lead to obtaining a higher percentage of cells that have a higher concentration of chlorophyll instead of a high percentage of cells that have a high concentration of astaxanthin during cell sorting using FACS. While the above specifically describes H. pluvialis as a particular example of a cell that may be astaxanthin rich or chlorophyll rich based on the cell culture conditions, it should be understood the operations of the method 400 may be used for astaxanthin rich cells of any other cell population (e.g., algae, fungi, bacteria etc.) from chlorophyll rich or other autofluorescent compound rich cells of the same population.

The method 400 provides a novel process for separating astaxanthin rich cells, for example cells having a higher concentration of astaxanthin relative to chlorophyll from chlorophyll rich cells, for example cells having a higher concentration of chlorophyll relative to astaxanthin. The method 400 includes providing a cell culture at operation 402. A first portion of the cells included in the cell culture have a high concentration of astaxanthin relative to chlorophyll, and a second portion of the cells in the cell culture have a high concentration of chlorophyll relative to astaxanthin. For example, the cell culture may include a plurality of H. pluvialis cells. A first portion of the H. pluvialis cells may be astaxanthin rich and a second portion of the H. pluvialis cells may be chlorophyll rich.

A laser having a first wavelength that distinguishes between a chlorophyll fluorescence signal and an astaxanthin fluorescence signal is determined at operation 404. For example, the laser that can best separate a chlorophyll fluorescence signal vs. astaxanthin fluorescence signal may be determined using a confocal scanning microscope (e.g., a Leica SPE confocal scanning microscope) with an acousto-optical tunable filter (AOTF). The confocal microscope may allow determination of the optimal excitation line or wavelength and fluorescence emission window to distinguish astaxanthin vs. chlorophyll.

In some embodiments, a mixture of reddened and green cells may be positioned in the same microscopic field for the testing to determine band pass settings that may effectively reverse the strength of emission signals from the two types of cells. In particular embodiments, the first wavelength of the laser that may optimally distinguish between chlorophyll and astaxanthin is in the range of 403 nm to 409 nm (e.g., 403, 404, 405, 406, 407, 408 or 409 nm). For example, while both chlorophyll and astaxanthin have fluorescence emission peaks or signals at 675 nm under 488 nm excitation, the astaxanthin fluorescence signal from the astaxanthin rich cells may shift its emission peak closer to 650 nm under excitation from the laser having the first wavelength in the range of 403 nm to 409 nm (e.g., 405 nm or 407 nm). In contrast, the chlorophyll fluorescence signal from the chlorophyll rich cells may maintain their emission peak at 675 nm under excitation from the laser having the first wavelength. Thus, the laser having the first wavelength allows separation of the emission peaks of the chlorophyll fluorescence signal and astaxanthin fluorescence signal. In other embodiments, lasers having wavelengths in the range of 350 nm to 460 nm (e.g., 350, 360, 370, 380, 390, 400, 410, 420, 430, 440, 450 or 460 nm inclusive of all ranges and values therebetween, or any other suitable laser may be used.

The cells included in the cell sorter are analyzed or communicated through a FACS at operation 406. The FACS can include any of the FACS described previously herein which includes the laser having the first wavelength (e.g., 407 nm). The cells included in the cell are excited using the laser at operation 408. The excitation causes the cells to produce a fluorescence signal. The fluorescence signal includes the chlorophyll fluorescence signal and the astaxanthin fluorescence signal.

The fluorescence signal is passed through a first channel including a first band pass filter and a second channel including a second band pass filter at operation 410. For example, the fluorescence signal may be split (e.g., by a beam splitter) into a fluorescence signal first portion which is communicated through the first channel and a fluorescence signal second portion which is communicated through the second channel. The first band pass filter may allow a first portion of the fluorescence signal having a wavelength in the range of 650 nm to 670 nm corresponding to the emission peak of the astaxanthin fluorescence signal to pass therethrough. Furthermore, the second band pass filter may allow a second portion of the fluorescence signal having a wavelength in the range of 685 nm to 735 nm corresponding to the emission peak of the chlorophyll fluorescence signal to pass therethrough. The opposite arrangement of the first and second filters may also be implemented.

A ratio of the first portion of the fluorescence signal of each cell that passed through the first channel to the second portion of the fluorescence signal that passed through the second channel is determined at operation 412. In response to the ratio being higher than the predetermined threshold, it is determined that the cell has a higher concentration of astaxanthin relative to chlorophyll at operation 414. The first portion of the cells having the higher concentration of astaxanthin relative to chlorophyll are separated from the second portion of the cells at operation 416.

Expanding further, a 407 nm violet laser may be used to excite the cells and the fluorescence signal or emissions from each cell passed through the first channel and the second channel. Cells that emit highly in both signals may be largely green, i.e., having a higher concentration of chlorophyll relative to astaxanthin. In this scenario the ratio may be about 1. In contrast, cells having a higher concentration of astaxanthin relative to chlorophyll may have higher emissions in the first channel relative to the second channel such that the ratio is greater than 1. In some embodiments, the predetermined threshold may be set at 1.5, 2, 3 or 4 inclusive of all ranges and values therebetween so as to separate cells having a substantially higher concentration of astaxanthin relative to chlorophyll (e.g., 1.5 fold, 2 fold, 3 fold, 4 fold or even higher) from the cell culture. In this manner, the method 400 provides a stain free method of separating astaxanthin rich cells from chlorophyll rich cells.

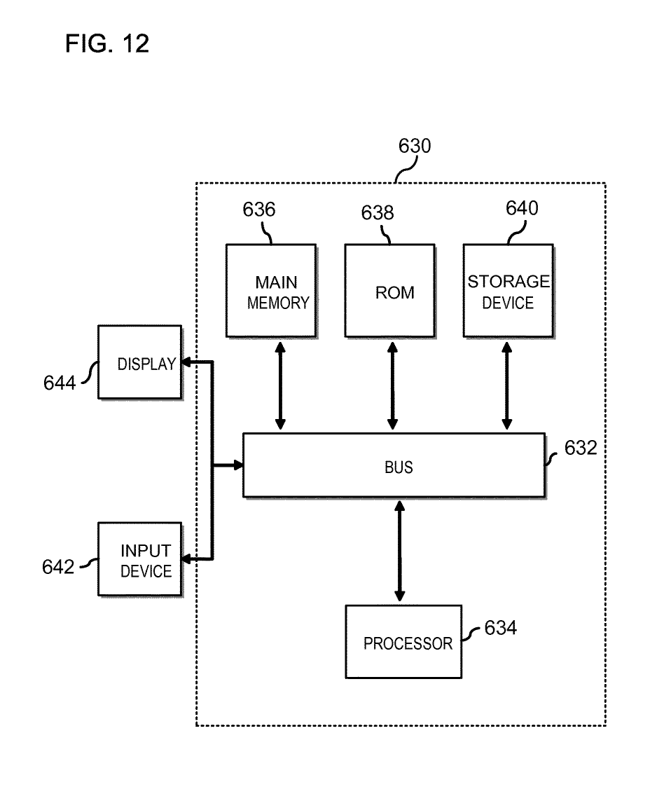

In some embodiments, operations of any of the methods described herein may be stored as instructions on a computer readable medium for execution by a computing device. In some embodiments, systems and methods described herein may include a computing device for performing operations of the various methods described herein. For example, FIG. 12 is a block diagram of a computing device 630 in accordance with an illustrative implementation. The computing device 630 can be used to perform any of the methods or the processes described herein, for example the method 100/200/300/400. The computing device 630 includes a bus 632 or other communication component for communicating information. The computing device 630 can also include one or more processors 634 or processing circuits coupled to the bus 632 for processing information.

The computing device 630 also includes main memory 636, such as a random access memory (RAM) or other dynamic storage device, coupled to the bus 632 for storing information, and instructions to be executed by the processor 634. Main memory 636 can also be used for storing position information, temporary variables, or other intermediate information during execution of instructions by the processor 634. The computing device 630 may further include ROM 638 or other static storage device coupled to the bus 632 for storing static information and instructions for the processor 634. A storage device 640, such as a solid-state device, magnetic disk or optical disk, is coupled to the bus 632 for persistently storing information and instructions.

The computing device 630 may be coupled via the bus 632 to a display 644, such as a liquid crystal display, or active matrix display, for displaying information to a user. An input device 642, such as a keyboard or alphanumeric pad, may be coupled to the bus 632 for communicating information and command selections to the processor 634. In another implementation, the input device 642 has a touch screen display 644.

According to various implementations, the processes and methods described herein can be implemented by the computing device 630 in response to the processor 634 executing an arrangement of instructions contained in main memory 636 (e.g., the operations of the method 100/200/300/400). Such instructions can be read into main memory 636 from another non-transitory computer-readable medium, such as the storage device 640. Execution of the arrangement of instructions contained in main memory 636 causes the computing device 630 to perform the illustrative processes described herein. One or more processors in a multi-processing arrangement may also be employed to execute the instructions contained in main memory 636. In alternative implementations, hard-wired may be used in place of or in combination with software instructions to effect illustrative implementations. Thus, implementations are not limited to any specific combination of hardware and software.

Although an example computing device has been described in FIG. 12, implementations described in this specification can be implemented in other types of digital electronic, or in computer software, firmware, or hardware, including the structures disclosed in this specification and their structural equivalents, or in combinations of one or more of them.

Implementations described in this specification can be implemented in digital electronic, or in computer software, firmware, or hardware, including the structures disclosed in this specification and their structural equivalents, or in combinations of one or more of them. The implementations described in this specification can be implemented as one or more computer programs, i.e., one or more circuitries of computer program instructions, encoded on one or more computer storage media for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate components or media (e.g., multiple CDs, disks, or other storage devices). Accordingly, the computer storage medium is both tangible and non-transitory.

The operations described in this specification can be performed by a data processing apparatus on data stored on one or more computer-readable storage devices or received from other sources. The term “data processing apparatus” or “computing device” encompasses all kinds of apparatus, devices, and machines for processing data, including by way of example a programmable processor, a computer, a system on a chip, or multiple ones, or combinations of the foregoing. The apparatus can include special purpose logic, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a circuitry, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more circuitries, sub-programs, or portions of code). A computer program can be deployed to be executed on one computer or on multiple computers that are located at one site or distributed across multiple sites and interconnected by a communication network.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings and tables in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated in a single software product or packaged into multiple software products.

Thus, particular implementations of the invention have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

As used herein, the singular forms “a”, “an” and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes and omissions may also be made in the design, operating conditions and arrangement of the various exemplary embodiments without departing from the scope of the present invention.

Reference throughout this specification to “one embodiment,” “an embodiment,” or similar language means that a particular feature, structure, or characteristic described in connection with the embodiment is included in at least one embodiment of the present disclosure. Appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the present disclosure, however, absent an express correlation to indicate otherwise, an implementation may be associated with one or more embodiments.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings and tables in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated in a single software product or packaged into multiple software products.

Thus, particular implementations of the invention have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

1-6. (canceled)

7. A method of dual staining cells for comparing a first culture of control cells and a second culture of experimental cells, comprising:
diluting the first culture of control cells to a first concentration;
diluting the second culture of experimental cells to a second concentration, the second concentration equal to the first concentration;
reducing the temperature of the first culture and the second culture to a first temperature;
adding a volume of a first staining solution to one of the first culture and the second culture, the first staining solution including a concentration of a first dye dissolved in a first concentration of DMSO;
adding a second volume of water to one of the first culture and the second culture which does not include the first staining solution; the second volume equal to the first volume;
incubating each of the first culture and the second culture for a first time;
mixing the first culture and the second culture to form a mixed culture;
adding a volume of a second staining solution to the mixed culture, the second staining solution including a concentration of a second dye dissolved in a second concentration of DMSO,
wherein, greater than 90% of the experimental cells and control cells remain alive in the mixed culture after the dual staining.

8. The method of claim 7, wherein each of the first concentration of the first culture and the second concentration of the second culture includes 5.0×10⁶ cells/milliliter.

9. The method of claim 7, wherein the first temperature is less than 15 degrees Celsius.

10. The method of claim 7, wherein the first dye included in the first staining solution is SYTO 84.

11. The method of claim 7, wherein the second dye includes BODIPY 505/515.

12. The method of claim 7, wherein each of the first concentration of DMSO and the second concentration of DMSO is in the range of 0.5% to 1% by volume.

13. A method of sorting cells on a fluorescence assisted cell sorter (FACS), comprising:
providing a culture of cells stained with a dye;
exciting the dye using photons at a first wavelength;
collecting fluorescence and emission of the dye at a second wavelength;
producing droplets of the cells by pumping the cell culture at a first pressure through a nozzle having a nozzle diameter;
producing droplets at a first frequency; and
sorting cells by a desired property.

14. The method of claim 13, further comprising:
setting a window gate extension to 0.5.

15. The method of claim 13, further comprising:
setting a threshold forward-scattered light to less than 8,000.

16. (canceled)

17. The method of claim 13, wherein the sorting the cells by a desired property includes sorting the cells by size using at least one of a forward scatter area (FSC-A), a forward scatter height (FSC-H), a forwards scatter width (FSC-W), side scatter area (SSC-A), a side scatter height (SSC-H) and a side scatter width (SSC-W) of the fluorescence of the dye to determine a size of the cells.

18. The method of claim 13, wherein the cells are sorted by size using a first gate, a second gate and a third gate:
wherein, the first gate sorts the cells by comparing the FSC-A versus the SSC-A,
wherein the second gate sorts the cells by comparing the FSC-H versus the FSC-W, and
wherein, the third gate sorts the cells by comparing the SSC-H versus the SSC-W.

19. The method of claim 14, wherein the first gate sorts for a first portion of the cells which includes a top 2.5% of the cells based on fluorescence, the second gate sorts for a second portion of the cells which includes 2.5% of the cells subsequent to the first portion based on fluorescence, and the third gate sorts for a third portion of the cells which includes 2.5% of the cells subsequent to the second portion based on fluorescence.

20. The method of claim 15, wherein the cells include algal cells, the method further comprising:
sorting the cells by chlorophyll content using a fourth gate, wherein the fourth gate sorts cells by comparing a PerCP-Cy5.5 height (PerCP-H) versus a PerCP-Cy5.5 width (PerCP-W).

21. The method of claim 13, wherein the dye includes BODIPY 505/515.

22. (canceled)

23. The method of claim 21, wherein the second wavelength is in the range of 505 nm to 515 nm.

24. The method of claim 13, wherein the first pressure is about 70 psi.

25. The method of claim 13, wherein the nozzle diameter is about 70 microns.

26. The method of claim 13, wherein the first frequency is about 7,500 Hz.

27. A method for sorting astaxanthin rich cells from chlorophyll rich cells, comprising:
providing a cell culture, a first portion of the cells included in the cell culture having a high concentration of astaxanthin relative to chlorophyll, and a second portion of the cells included in the cell culture having a high concentration of chlorophyll relative to astaxanthin;
determining a laser having a laser wavelength that distinguishes between a chlorophyll fluorescence signal and an astaxanthin fluorescence signal;
communicating the cells included in the cell culture through a fluorescence assisted cell sorter (FACS);
exciting the cells included in the cell culture with the laser, the exciting causing each of the cells included in the cell culture to produce a fluorescence signal, the fluorescence signal comprising the chlorophyll fluorescence signal and the astaxanthin fluorescence signal;
passing the fluorescence signal through a first channel comprising a first band pass filter, and a second channel comprising a second band pass filter;
determining a ratio of a first portion of the fluorescence signal of each cell that passed through the first channel to a second portion of the fluorescence signal that passed through the second channel; and
in response to the ratio being higher than the predetermined threshold, determining that the corresponding cell has a high concentration of astaxanthin; and
sorting the first portion of the cells having the high concentration of astaxanthin relative to chlorophyll from the second portion of the cells.

28. The method of claim 18, wherein the laser wavelength is in the range of 403 nm to 409 nm, the first band pass filter is in the range of 650 nm to 670 nm, and the second band pass filter is in the range of 685 nm to 735 nm.

29. The method of claim 19, wherein the cell culture includes cells of the algae Haematococcus pluvialis.
